(19) United States
(12) Patent Application Publication
(10) Pub. No.: US 2026/0338647 A1
(43) Pub. Date: Oct. 29, 2026

(54) RECEPTACLE FOR A SAMPLING AND/OR MEASURING DEVICE

(71) Applicant: Vapor Pin Enterprises, Inc., Plain City, OH (US)

(72) Inventor: Craig A. Cox, Columbus, OH (US)

(21) Appl. No.: 19/120,601

(22) PCT Filed: Aug. 14, 2023
(86) PCT No.: PCT/US2023/072159

§ 371 Date: Apr. 11, 2025

Related U.S. Application Data
(60) Provisional application No. 63/379,283, filed on Oct. 12, 2022.

(51) Int. Cl. G01N 1/22; G01N 33/00

(52) U.S. Cl. CPC G01N 1/2294; G01N 33/0009

(57) ABSTRACT

A receptacle for a sampling and/or measuring device for sub-slab soil fluid having a housing portion and a sieve portion. The housing portion may be configured to be releasably coupled to an adapter body of a fluid sampling and/or measuring system. An internal cavity formed in the housing portion has a suitable shape, size, and configuration to receive a sampling and/or measuring device therein. The sieve portion may be configured to be releasably coupled to the housing portion. The sieve portion permits a flow of a fluid into the receptacle, while maintaining a position of the sampling and/or measuring device therein. The sieve portion may further include one or more lateral openings formed therein.

### CROSS-REFERENCE TO RELATED APPLICATION

This application claims the benefit of U.S. Provisional Patent Application Ser. No. 63/379,283, filed Oct. 12, 2022, the entirety of which is herein incorporated by reference.

### FIELD

Exemplary embodiments are directed to mechanical devices and systems. More particularly, exemplary embodiments are directed to a device and system for facilitating a sampling and/or measuring of a fluid.

### BACKGROUND

The potential for volatile organic compounds (VOCs) associated with contaminated soil and groundwater to enter homes and businesses through basements and building slabs is a recent focus of federal and state environmental protections agencies. This potential route of exposure is commonly referred to as the “vapor intrusion pathway.” Evaluations of the potential risk associated with long-term exposure to VOCs have been published by the United States Environmental Protection Agency (EPA) and other entities. These evaluations indicate that very low concentrations of some of these VOCs, on the order of a few parts per billion in some cases, can pose an unacceptable risk to building occupants. In some situations, sub-slab soil fluid samples are collected to evaluate vapor concentrations and the potential for these vapors to enter a building.

The science of analyzing samples of sub-slab soil fluid is known. However, the practicalities of collecting these samples of fluid are quite cumbersome. Techniques and devices currently used and proposed in recent draft guidance documents by the EPA and other agencies to collect sub-slab soil fluid samples are built upon the experience of environmental professionals gained over many years of sampling groundwater via monitor wells. In essence, the current state of the art for sub-slab sampling is the use of a miniature well installed through the slab. These wells, or “sub-slab vapor points” are typically installed by boring a fairly crude hole through the slab and cementing a metal tube in place. At the top of the tube are a number of threaded fittings that allow the vapor point to be connected via plastic tubing to an evacuated vessel, known in the art as a summa canister.

Because the levels of concern for many of the VOCs are so low, leaks in the vapor point fittings or along the edge of the vapor point itself allow indoor air to dilute the sample, rendering the sample useless. This situation is exacerbated by the fact that most vapor points must be sampled on multiple occasions. Each time the vapor point is used it must be disconnected and reconnected using multiple wrenches, usually in tight quarters. This activity can cause some fittings to progressively loosen and leak more readily, or result in the point itself losing its bond with the cement used to anchor it during installation. Federal and state EPA officials recognize this shortcoming and have developed elaborate, time consuming methods for detecting such leaks.

The collection of sub-slab samples can also be inconvenient to building occupants since it requires the removal of floor coverings and coring or drilling of the foundation slab. One recommended method is using an electric hammer drill or rotary hammer to produce an inner pilot hole into the concrete slab. After the pilot hole is drilled, an individual must drill an outer hole to a predetermined depth using a larger drill bit. After the outer hole is finished, the individual must use the original tool to assure that the pilot hole is then drilled through the slab and several inches into the sub-slab material. Once the drilling is completed, a stainless steel probe is assembled and inserted into the pre-drilled hole. The probe is mounted as flush as possible with the surrounding slab to minimize the interference with pedestrian or vehicular traffic. The probe has to be cemented into place to ensure that the probe assembly is air-tight with the foundation slab. Since the cement has to cure, an individual must come back at least one further time before sampling of the sub-soil may occur, further inconveniencing a homeowner or business.

Attempts have been made to overcome these and other difficulties inherent in the task of collecting sub-slab soil fluid samples for analysis. Various devices and systems have been developed for use in such collection, for instance those previously described in U.S. Pat. Nos. 8,220,347 and 9,291,531, both co-owned by the applicant and the fully incorporated herein by reference. Those references disclose devices, systems and their methods of use that facilitate the collection of sub-slab soil fluid samples by, in part, eliminating the intrusion of the collection system on the interior building space, reducing the potential for damage to the slab introduced by previously used methods of collection, reducing or eliminating the risk of leakage during sampling thereby increasing testing efficacy/efficiency, and reducing collection costs through the introduction of reusable system components, for instance.

However, it has been found that certain disadvantages and drawbacks remain in the current state-of-the-art devices and systems. For example, variations in slab, bedding and foundation thicknesses, and in geographic structures of various testing locations have resulted in a need for sub-slab soil fluid collection at variable depths relative to the top surface of a particular slab. Furthermore, as the art of sub-slab soil fluid analysis continues to advance, soil fluid collection may be needed at an increasing variety of depths relative to the top surface of a given slab. In some instances, drilling well into the backfill or native material beneath a slab to a desired depth for collection is found to increase the potential for clogging or the introduction of undesirable particulates into the vapor stream entering the sampling device.

In some cases, it may be desirable to introduce at least one sampling and/or measuring device or probe into a space beneath a slab. Currently known systems, however, either are not compatible with such sampling and/or measuring devices or require invasive installation techniques that are cumbersome, undesirable, and often cause unwanted damage to the slab or structure.

Devices and systems that eliminate some or all of the drawbacks of the known devices and techniques for measuring sub-slab soil fluid are desired. Providing a leak-resistant device that allows for prompt installation and removal, saving time and money may eliminate some or all of these drawbacks. Also, a device and system that allows for installation to occur in one appointment is desirable. Such a device may also be designed for use with different VOC measuring devices, both above-and below-slab, and with other sampling and/or measuring devices generally. There is also need for a device and system that provides some or all of these advantages in addition to the ability to collect samples and/or measurements at a point beneath the slab, and without clogging or contamination of the device and sample, respectively.

### SUMMARY

In concordance and agreement with the presently described subject matter, device and system for facilitating a sampling and/or measuring of sub-slab soil fluid wherein a size, weight, cost, and complexity thereof is minimized, while optimizing a performance thereof, have surprisingly been discovered.

Exemplary embodiments of a device and a system for facilitating a sampling of sub-slab soil fluid may eliminate some or all of the aforementioned drawbacks of the current art are illustrated in the figures and described hereinafter.

In one embodiment, a receptacle, comprises: a housing portion having a cavity configured to receive at least one sampling and/or measuring device for a sub-structure fluid.

In another embodiment, a system for facilitating a sampling and/or measuring of a sub-structure fluid, comprises: a receptacle including a housing portion and a sieve portion configured to be coupled to the housing portion, the housing portion having a cavity configured to receive at least one sampling and/or measuring device for the sub-structure fluid.

As aspects of some embodiments, the cavity is configured to permit a flow of the sub-structure fluid therethrough.

As aspects of some embodiments, the cavity has a first diameter and a second diameter, and wherein the second diameter is greater than the first diameter.

As aspects of some embodiments, the first diameter is substantially equal to a diameter of a cavity formed in an adapter body.

As aspects of some embodiments, the housing portion includes a first end, a second end, and an intermediate segment formed therebetween.

As aspects of some embodiments, the cavity extends along a longitudinal axis of the housing portion from the first end to the second end.

As aspects of some embodiments, at least one of the first end and the second end is provided with one or more coupling features.

As aspects of some embodiments, the first end is configured to be coupled to an adapter body and the second end is configured to be coupled to the sieve portion of the receptacle.

As aspects of some embodiments, the sieve portion includes a cavity formed therein to permit a flow of the sub-structure fluid therethrough.

As aspects of some embodiments, the sieve portion includes one or more lateral openings formed therein to permit a flow of the sub-structure fluid therethrough.

In yet another embodiments, a method of sub-structure fluid testing, comprises: providing a receptacle including a housing portion having a cavity formed therein; disposing at least one sampling and/or measuring device into the cavity of the housing portion; disposing the receptacle into a structure until at least a portion of the receptacle extends beyond a lower surface thereof; permitting a flow of fluid through the receptacle; and removing the receptacle from the structure to obtain the at least one sampling and/or measuring device therefrom.

As aspects of some embodiments, the method further comprises coupling a sieve portion of the receptacle to the housing portion after the at least one sampling and/or measuring device is disposed therein and prior to disposing the receptacle into the structure.

As aspects of some embodiments, the receptacle is coupled to an adapter body, and wherein a proximal end of the adapter body is at least flush with an upper surface of the structure when the receptacle is disposed in the structure.

It is an object of this present disclosure to provide a system for use in the collection of sub-slab soil fluid of the type generally described herein, being adapted for the purposes set forth herein, and overcoming disadvantages found in the prior art.

These and other advantages are provided by the present disclosure described and shown in more detail below.

### BRIEF DESCRIPTION

In addition to the features mentioned above, other aspects of the present disclosure will be readily apparent from the following descriptions of the drawings and exemplary embodiments, wherein like reference numerals across the several views refer to identical or equivalent features, and wherein:

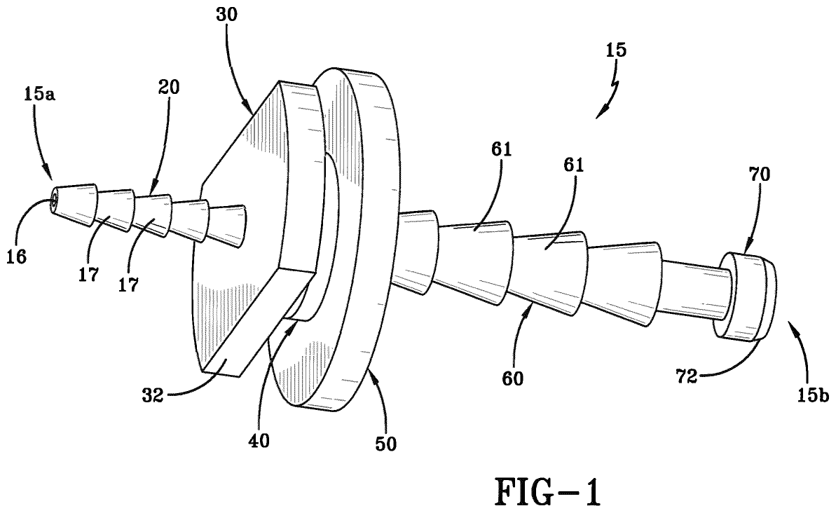

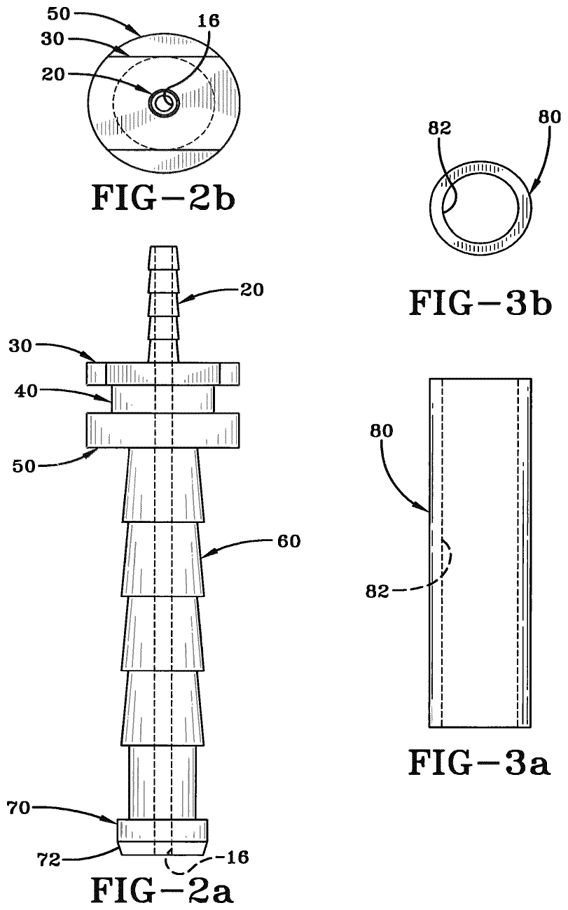

FIG. 1 is a perspective view illustrating an exemplary embodiment of an adapter body;

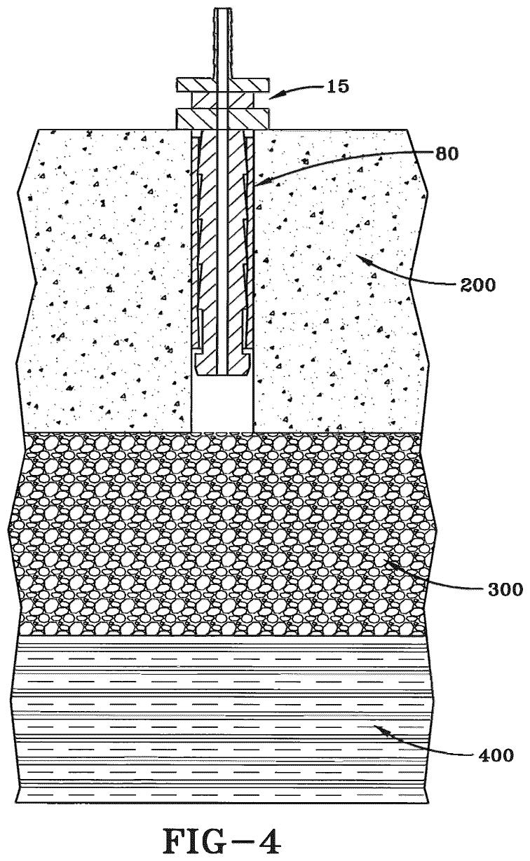

FIG. 2a is a front elevation view of the adapter body of FIG. 1;

FIG. 2b is a top plan view of the adapter body of FIGS. 1 and 2a;

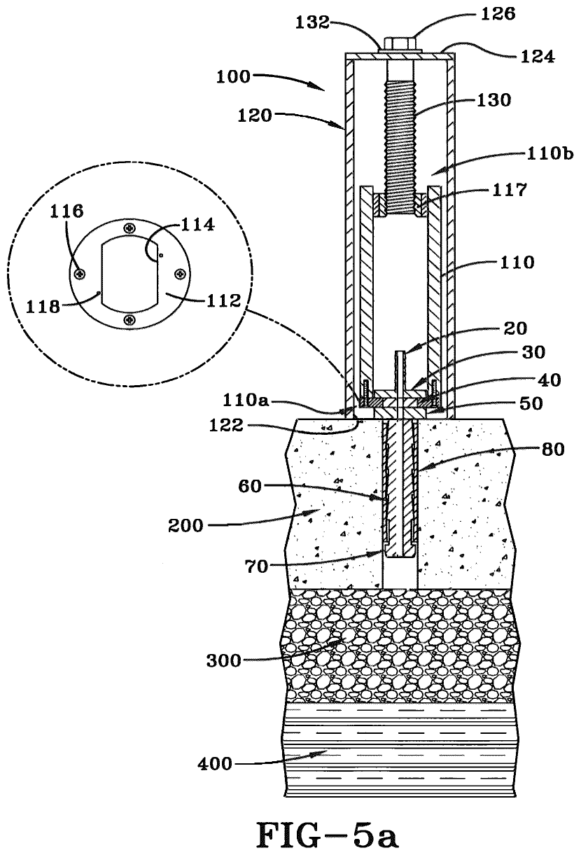

FIG. 3a is a front elevation view of an exemplary embodiment of a tubular body;

FIG. 3b is a top plan view of the tubular body of FIG. 3a;

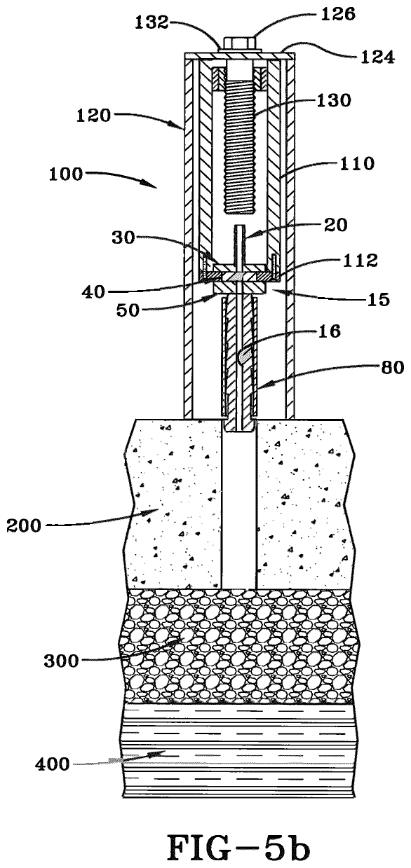

FIG. 4 is a sectional view of an exemplary embodiment of an adapter body and tubular body installed within a foundation slab;

FIG. 5a is a sectional view of the adapter body and tubular body of FIG. 4 with the installation tool prior to extraction;

FIG. 5b is a sectional view thereof after extraction has occurred;

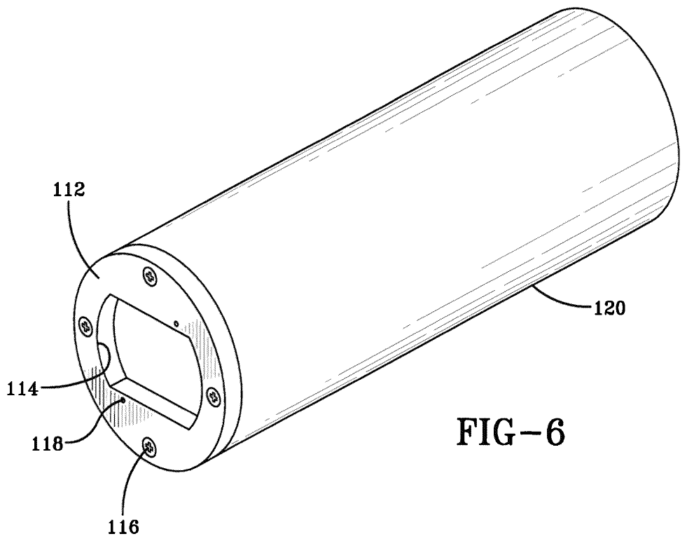

FIG. 6 is a perspective view of an exemplary embodiment of an installation tool;

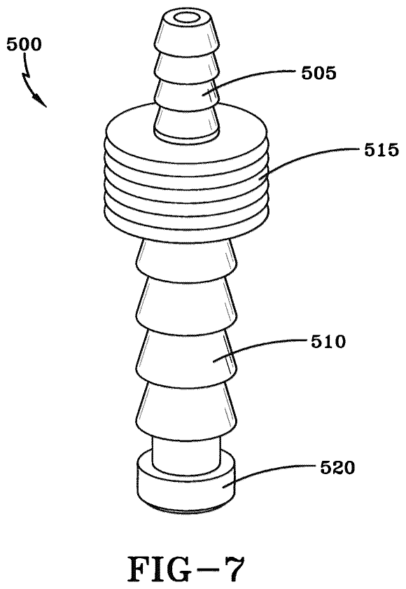

FIG. 7 is a front perspective view of a further exemplary embodiment of an adapter body;

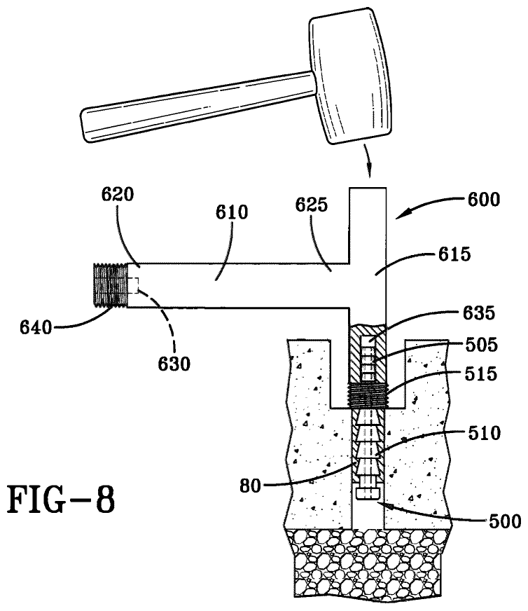

FIG. 8 is a side view of an exemplary tool being used to install the adapter device and tubular body of FIG. 7;

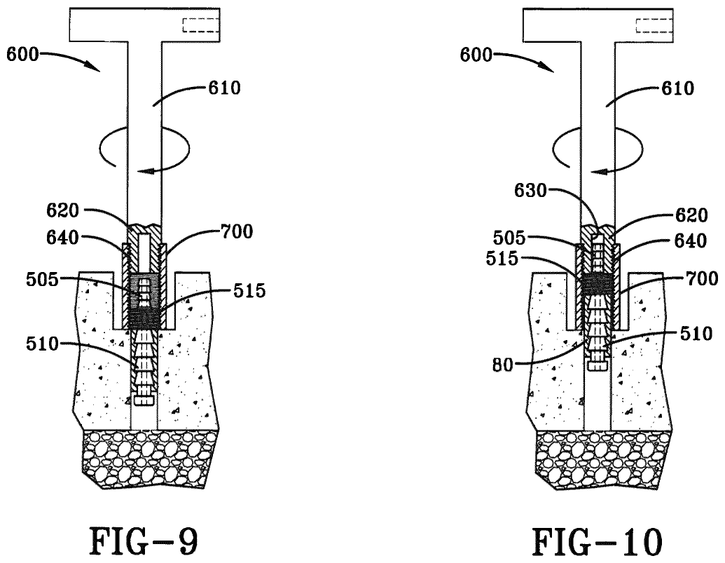

FIG. 9 illustrates the tool of FIG. 8 being used to remove adapter body and tubular body of FIG. 7;

FIG. 10 illustrates a further view of the extraction process of the adapter body and tubular body of FIG. 7;

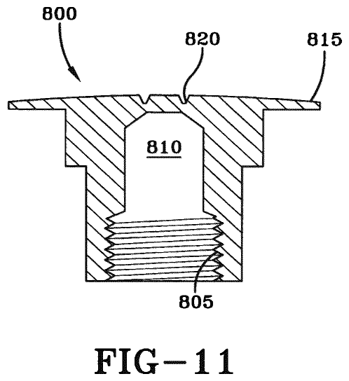

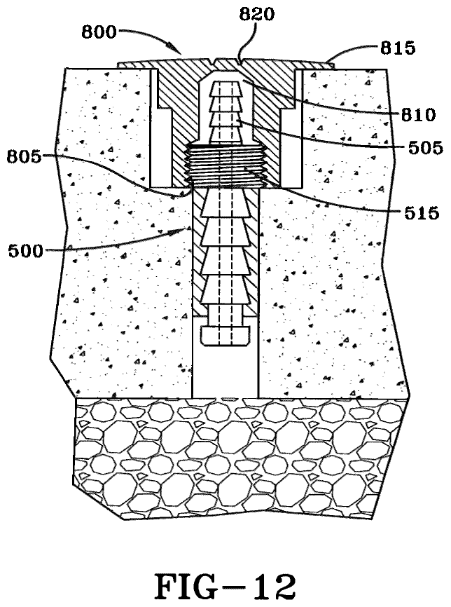

FIG. 11 is a sectional view of an exemplary covering for exemplary embodiments of the adapter body;

FIG. 12 is a sectional view of an exemplary covering engaged with an exemplary adapter body installed in a foundation slab;

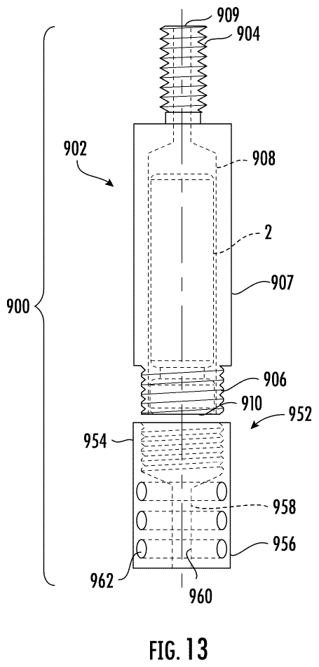

FIG. 13 is an exploded, front elevational view of an exemplary embodiment of a receptacle for a sampling and/or measuring device, wherein the receptacle comprises a housing portion and a sieve portion;

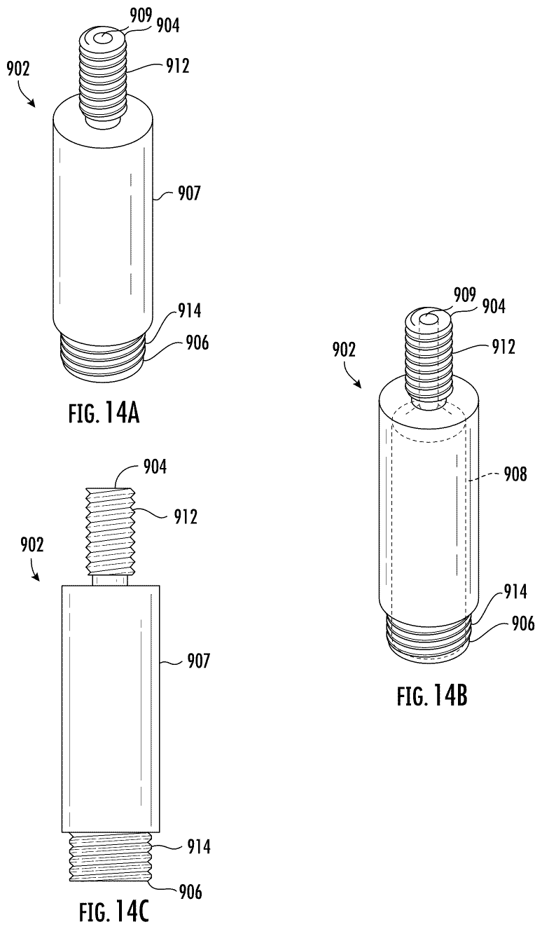

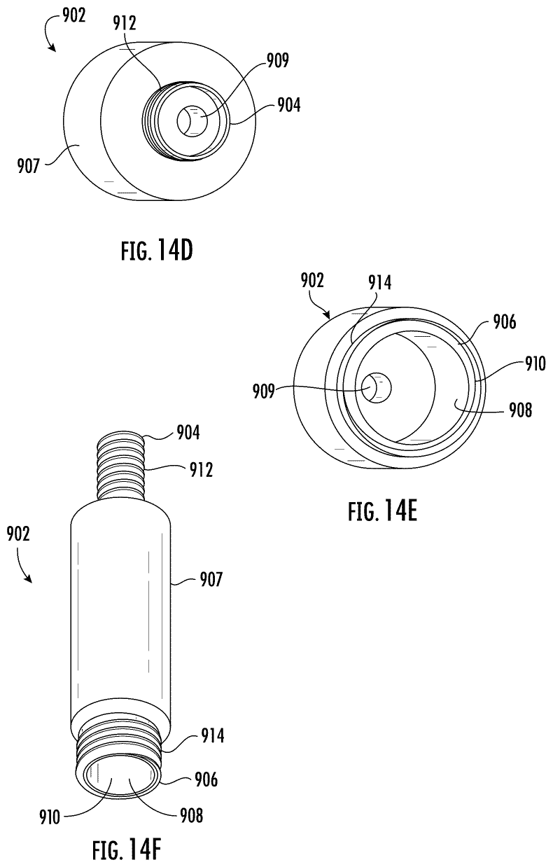

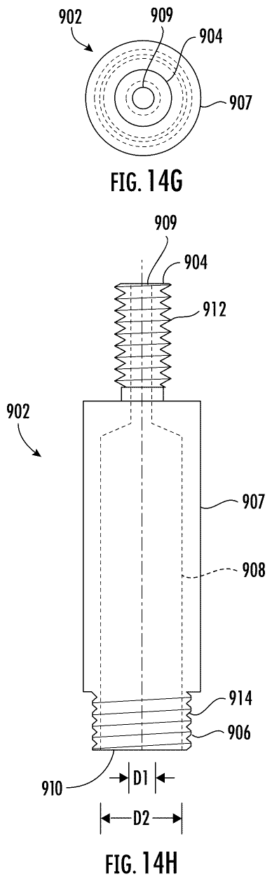

FIG. 14A is a front perspective view of an exemplary embodiment of the housing portion of the receptacle illustrated in FIG. 13;

FIG. 14B is a front perspective view of an exemplary embodiment of the housing portion of FIG. 14A, wherein an internal cavity is shown in dashed lines;

FIG. 14C is a front elevational view of the housing portion of FIGS. 14A and 14B;

FIG. 14D is a top perspective view of the housing portion of FIGS. 14A-14C;

FIG. 14E is bottom perspective view of the housing portion of FIGS. 14A-14D;

FIG. 14F is a front perspective view of the housing portion of FIGS. 14A-14E;

FIG. 14G is a top plan view of the housing portion of FIGS. 14A-14F;

FIG. 14H is a front elevational view of the housing portion of FIGS. 14A-14G, wherein the internal cavity is shown in dashed lines;

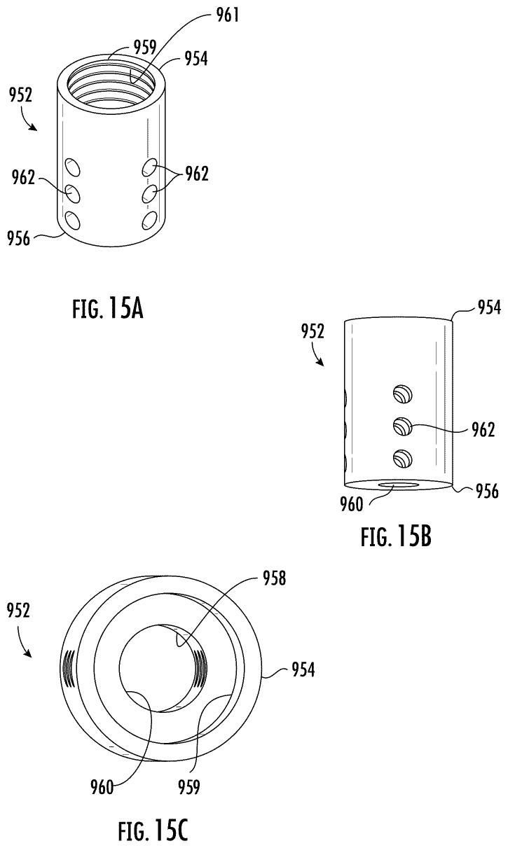

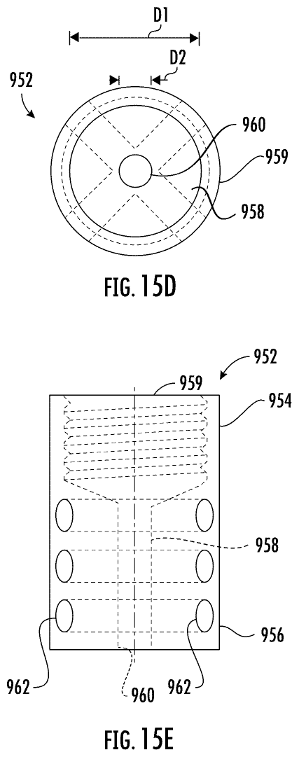

FIG. 15A is a front perspective view of an exemplary embodiment of the sieve portion of the receptacle illustrated in FIG. 13;

FIG. 15B is a side perspective view of the sieve portion of FIG. 15A;

FIG. 15C is a top perspective view of the sieve portion of FIGS. 15A and 15B;

FIG. 15D is a top plan view of the sieve portion of FIGS. 15A-15C;

FIG. 15E is a front elevational view of the sieve portion of FIGS. 15A-15D; and

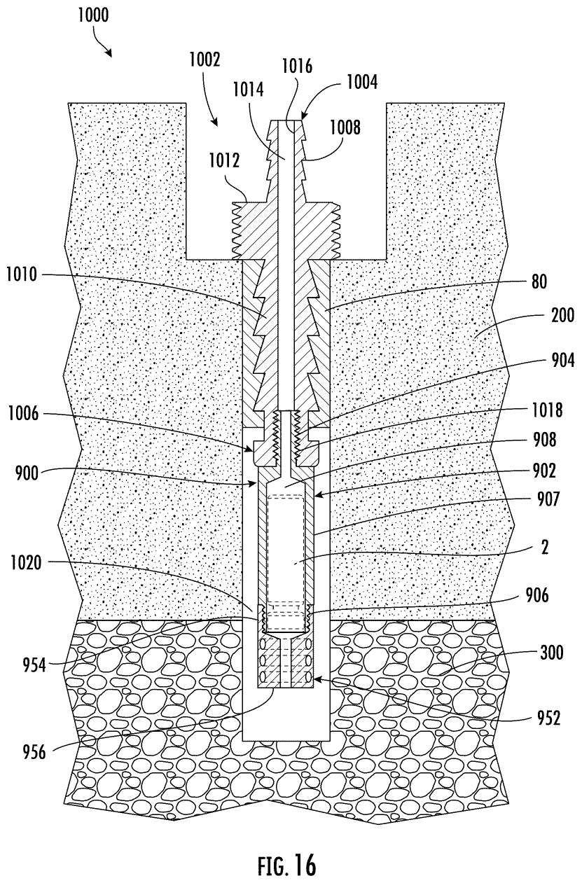

FIG. 16 is a cross-sectional view of an exemplary embodiment of a system for facilitating a sampling of sub-slab soil fluid comprising an adapter body and a receptacle for a sampling and/or measuring device, wherein the system is in use in connection with an exemplary slab and the receptacle has an exemplary sampling device disposed therein.

### DETAILED DESCRIPTION

The following description of technology is merely exemplary in nature of the subject matter, manufacture and use of the subject matter, and is not intended to limit the scope, application, or uses of any specific subject matter claimed in this application or in such other applications as may be filed claiming priority to this application, or patents issuing therefrom. Regarding methods disclosed, the order of the steps presented is exemplary in nature, and thus, the order of the steps can be different in various embodiments. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

All documents, including patents, patent applications, and scientific literature cited in this detailed description are incorporated herein by reference, unless otherwise expressly indicated. Where any conflict or ambiguity may exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

FIG. 1 depicts one exemplary embodiment of an adapter body 15. As shown, this particular adapter body 15 includes a first barbed portion 20, an external engaging portion 30, a recess 40, a collar portion 50, a second barbed portion 60 and a raised end 70.

As shown in FIGS. 1-2b, the adapter body includes a proximal end 15a and a distal end 15b. Exemplary embodiments of the adapter body 15 may include a first barbed portion 20, an external engaging portion 30, a recess 40, a collar portion 50, a second barbed portion 60 and a raised end 70. As seen in FIGS. 2a-2b, adapter bodies 15 include an internal cavity 16 that axially passes through the length of the adapter body 15 from the proximal end 15a to the distal end 15b. The internal cavity 16 allows fluid (e.g., a gas and/or a liquid) found in the subsoil to flow through the adapter body 15 and be read by one or more fluid sampling and/or measuring devices (e.g., an internal sampling and/or measuring device 2 depicted in FIG. 16) that is fluidly connected with the adapter body 15. The cross-sectional area and geometry of the internal cavity 16 may be substantially similar throughout the length of the adapter body 15.

In this embodiment, the first barbed portion 20 of the adapter body 15 is located towards the proximal end 15a thereof. The first barbed portion 20 generally includes at least one barb 17. In some examples, the barbs 17 are generally conical in geometry to facilitate the releasable securement of an exemplary embodiment of tubing (not shown) that connects the adapter body 15 with one or the sampling and/or measuring devices, such as a SUMMA canister. As such, the first barbed portion 20 is often manufactured from readily available sizes of round stock, thereby reducing manufacturing time and expense, although it may have any number of cross-sectional geometries depending upon the cross-sectional geometry of the tubing that connects the device with the fluid sampling and/or measuring device. Typically, the end-most barb located towards the proximal end 15a may include a generally rounded face that facilitates the insertion of the first barbed portion 20 within the inner cavity of the tubing that connects the adapter body 15 with a fluid sampling and/or measuring device. In some exemplary embodiments, there are no gaps or land sections between the barbs 17. In such embodiments, the end of the barb with the smaller outside diameter may abut the next barb's end with the larger outside diameter.

Typically, when the barbs 17 bear a fixed dimensional relationship to the inside diameter of the tubing that connects the adapter body 15 with a fluid sampling and/or measuring device, the tubing will form a reliable pressure tight seal to the adapter body 15. In one embodiment, the large diameter ends of the barbs 17 may be approximately 0.30″, while the inner diameter of the tubing may be approximately 0.25″. This type of press-fit may cause the tube to spread or flare so that after the first barbed portion 20 is fully inserted within the tube, the tube will return to its original size after releasable securement. Furthermore, in some embodiments, the conical shape of the barb 17, which is wider toward the point of insertion, provides a manner of anchoring the flexible tubing body 80 during the insertion process so that the tubing body 80 does not move in relation to the adapter body 15 during insertion (see FIGS. 3a and 3b).

The external engaging portion 30 of the adapter body 15 includes an external engaging portion, in this example, a flange 32 adapted to engage a wrench or other tool. The external engaging portion 30 is shown here to be of substantially circular shape, wherein a portion of opposed sides are substantially parallel to one another. However, other shapes are also possible. In another example, the outside geometry of the external engaging portion 30 is substantially hexagonal or square in geometry to allow a user to engage thereto with a wrench or other tool. While this embodiment of the fastener engaging portion contains a flange, other embodiments include a component, which allows for engagement with different tools, including a screwdriver head component, a hex head component, TORX head component, drill head component, or another engaging structure that can tighten and/or move the adapter body 15 by rotational movement.

In some embodiments, the engaging portion 30 may be integral with the first barbed portion 20, such as by molding or turning. In other embodiments, the engaging portion 30 may be attached to the first barbed portion 20, such as by welding. Alternatively, the first barbed portion 20 may be removably attached to the engaging portion 30 so that the device 15 may be used with tubing of various sizes.

In exemplary embodiments, the collar portion 50 is generally joined to the engaging portion 30 by an optional recess area 40 which has a generally cylindrical shape. The geometry of the recess area 40 may be of various cross-sectional areas, although a substantially round cross-sectional area may simplify manufacturing. The optional recess area 40 may also allow a wrench or other tool 100 to engage the engaging portion 30 and/or the collar portion 50 of the adapter body 15 to facilitate the installation and/or removal of the adapter body 15. In one example, as seen in FIGS. 5a and 5b, an individual may use the tool 100 to install and/or remove the device.

In this example, the entire collar portion 50 is substantially circular in cross-sectional geometry, wherein the diameter is substantially the same along the length thereof. The cross-sectional geometry of the collar section is typically substantially circular to facilitate the insertion of adapter body 15 within in a corresponding hole in the slab that is likewise substantially circular. However, in other embodiments, the collar portion 50 may also be of other cross-sectional shapes. As aforementioned, one of the main functions of the collar portion 50 is to provide a surface for a tool to contact the adapter body 15 for installation and/or removal of the adapter body 15 during use. In some embodiments, during installation of the adapter body 15, once the distal end of the collar portion 50 engages a portion of the slab, the device is fully engaged, as depicted in FIG. 4. In some embodiments, the collar portion 50 may taper inward (not shown) from a larger diameter as it extends longitudinally from the proximal end 15a of the adapter body 15. The taper may facilitate the securement of the tubular body 80 to the adapter body 15 during installation. In some embodiments, the collar portion 50 may be integral with the engaging portion 30, and the recess portion 40 such as by molding or turning. In other embodiments, the engaging portion 30 and the collar portion 50 may be attached to the recess portion 40, such as by welding.

As shown in the illustration of an adapter body as depicted in FIGS. 1-2b, the second barbed portion 60 of the adapter body 15 may be located towards the distal end 15b thereof. The second barbed portion 60 generally includes at least one barb 61. In some examples, the barbs 61 are generally conical in geometry to facilitate the releasable securement of the tubing body 80, as seen in FIG. 4. As such, the second barbed portion 60 may be manufactured from readily available sizes of round stock, thereby reducing manufacturing time and expense. However, it should be realized that the second barbed portion 60 may have any number of cross-sectional geometries, depending upon the cross-sectional geometry of the tubular body 80. Typically, the barbs 61 may taper from a larger diameter from the distal end 15b thereof. However, in other embodiments, some or all of the barbs 61 may taper from a larger diameter from the proximal end 15a thereof. In some exemplary embodiments, there are no gaps or land sections between the barbs 61. In such embodiments, the end of the barb with the smaller outside diameter may abut the next barb's end with the larger outside diameter.

Typically, when the barbs 61 bear a fixed dimensional relationship to the inside diameter of the tubular body 80 there will form a reliable pressure tight seal therebetween. In one embodiment, the large diameter ends of the barbs 61 may be approximately 0.79″, while the inner diameter of the tubular body 80 may be approximately 0.75″. This type of press-fit may cause the tube to spread or flare so that after the second barbed portion 60 is fully inserted within the tubular body 80, the tubular body 80 will return to its original size after releasable securement.

The exemplary embodiment raised end 70 of FIG. 1 can be seen in more detail in FIG. 2a. As shown, the raised end 70 is a substantially cylindrical shape, although other shapes are possible. This example of the raised end include a chamfer 72 or rounded end located at the distal end 15b of the adapter body 15, which facilitates the insertion of the raised end 70 within the inner cavity of the tubular body 80. Typically, but not necessarily, the outside diameter of the raised end 70 is approximately the same diameter of the largest diameter of the barbs 61. However, in other embodiments, the outside diameter of the raised end 70 may be greater or less than the outside diameter of the barbs 61.

Adapter bodies may be made of any number of materials, such as, for example, brass, plastics, or other metals, such as stainless steel. Whatever material is selected, the resulting adapter body 15 should have sufficient strength to withstand the insertion and extraction of the adapter body within the slab. Furthermore, it is preferred that the material is easy to manufacture, if machined.

As shown in FIG. 4, during installation the second barbed portion 60 and raised end 70 has disposed thereon a tubular body 80. The tubular body 80 may be made of materials flexible enough to allow securement of the tubular body 80 around the second barbed portion 60 and the raised end 70, along with providing an air-tight seal between the adapter body 15 and the inside diameter of a hole drilled into the slab of a basement or foundation of a building. In one particular example, the tubular body 80 is fabricated from low-VOC content silicone tubing. As aforementioned, the interior cavity 82 of the tubular body 80 is adapted to receive the raised end 70 and second barbed portion 60 of the adapter body 15 and may be of any shape required to produce mating engagement therebetween. Furthermore, in some embodiments one or more optional seals (not shown) may be placed around the barbs 61 of the second barbed portion 60 to help effectuate an air-tight seal between the tubular body 80 and the adapter body 15. In some embodiments, it may be advisable to coat or otherwise cover the interior cavity of the tubular body 80 and/or the exterior of the second barbed portion 60 and/or raised end 70 with a high friction material for facilitating the engagement therebetween. Tubular body lengths may vary, depending upon the length between the collar portion 50 and the distal end 15b of the adapter body 15. In one example, the length of the tubular body 80 is approximately 3.75 inches. Likewise, the outside diameter of exemplary embodiments of the tubular body 80 may vary depending upon the inside diameter of the hole drilled or bored within the slab of concrete or other foundation of a building or other structure.

Particularly, in a normal assembled installation state as seen in FIG. 4, the tubular body 80 is wedged between the second barbed portion 60 and/or the raised end 70, and the inside wall of the drilled or cored hole that extends through the foundation slab. In some methods of installation, the tubular body 80 is releasably secured around the second barbed portion 60 before the device is installed within the cored hole. In other embodiments, an installation tool 100, as seen in FIGS. 5a and 5b may apply pressure on a portion of the adapter body 15 to effectuate installation within the cored hole.

During installation and/or extraction the tool 100 may include an inner body 110 that includes a contacting portion 112 at a first end 110a with an aperture 114 that complements the cross-sectional geometry of the engaging portion 30. In one example, the contacting portion 112 may be secured to the inner body 110 by one or more fasteners 116. However, in other examples the contacting portion 112 may be integral with the inner body 110, such as by welding, etc. The tool 100 may facilitate installation by allowing an individual to place the inner body 110 over and/or around the engaging portion 30 wherein at least a portion of the inner face of the contacting portion 112 of the tool 100 may contact the engaging portion 30 and/or at least a portion of the outer face of the contacting portion 112 may contact the collar portion 50 to allow the individual to strike a second portion of the tool 100 with a hammer or other object to facilitate installation of the adapter body 15.

In other embodiments, an installation tool 100, as seen in FIGS. 5a and 5b may apply pressure on a portion of the adapter body 15 to effectuate installation in and/or extraction from the cored hole. In this embodiment, the contacting portion 112 may be positioned over and around the engaging portion 30, wherein at least a portion of the inner face of the contacting portions 112 of the tool 100 may contact the engaging portion 30 and/or at least a portion of the outer face of the contacting portion 112 may contact the collar portion 50 when the inner body 110 is turned approximately ninety degrees. In some examples, a surface of the contacting portion 112 or inner body 110 may include one or more raised surfaces 118 or other stopping device adapted to prohibit an individual from turning the inner body 110 of the tool 100 beyond a desired location, to effectuate contact with the device for installation and/or removal.

Exemplary embodiments of the inner body 110 are tubular in cross-sectional geometry. In some examples, it may be preferred that the inner body 110 is substantially cylindrical. The inner body 110 may include a threaded surface 117 located towards a second end 110b. The threaded surface 117 may be integral with the inner body 110, or may be a separate piece adhered to within or to the inner body 110. The threaded surface 117 is adapted to complement the threaded surface of a bolt or other threaded fastener 130, described later and seen in FIGS. 5a and 5b.

In some examples, the tool 100 may further include an outer body 120 that is tubular in cross-sectional geometry. In the example depicted in FIGS. 5a and 5b, the outer body 120 is substantially cylindrical in cross-sectional geometry to complement the geometry of the inner body 110. The first end of the outer body 120 contains an aperture 122 large enough to allow the outer body 120 to be positioned around the inner body 110.

Furthermore, some exemplary embodiments of the outer body 120 may include a top portion 124 with an aperture 126 located towards the second end thereof. In the example depicted in FIGS. 5a and 5b, the top portion 124 is a plate adhered to the second end of the outer body 120. However, in other embodiments, the top portion 124 may be optionally secured with the outer body 120 by fasteners or other securing devices.

During one exemplary method of extraction of the adapter body 15, an individual may releasably secure the inner body 110 with the device as aforementioned. After the inner body 110 is secured with the adapter body 15, the individual may position the outer body 120 around the inner body 110, as depicted in FIGS. 5a and 5b, wherein at least a portion of the outer body 120 engages the concrete slab 200. The individual places a bolt or other threaded fastener 130 down through the aperture 126 located towards the second end. An optional washer 132 or similar device may be used to help distribute the force exerted on the head of the threaded fastener 130. An individual may then rotationally engage the threaded fastener 130 with the complementary threaded surface 117, effectuating the removal of the device, as seen in FIG. 5b.

Likewise, the complementary portion of the tool 100 may be placed over and around the engaging portion 30, then rotated approximately ninety degrees so that the adapter body 15 may be removed. In other embodiments, an installation tool 100, as seen in FIGS. 5a and 5b may apply pressure on a portion of the adapter body 15 to effectuate installation and/or removal from the cored hole.

In some installation methods, the adapter body is pressed downward in the cored hole until the collar engages the slab. However, some exemplary embodiments of the adapter body may install wherein the adapter body is mounted flush to accommodate a larger hole that is drilled deep enough to allow the first barbed portion to lie below the surface of the slab. In this exemplary embodiment, the entire adapter body is mounted at least flush, if not below the surface level of the slab, decreasing the likelihood that the device may be damaged after installation. Installation of exemplary embodiments of the adapter body may be installed into a five eighths-inch diameter hole cored through the slab of concrete or other foundation material. The cored hole provides a smoother bonding surface and can be accomplished using a standard, hand-held coring machine. Exemplary embodiments of the adapter body may be driven into the cored hole using a hammer or similar device.

Installation of exemplary embodiments of the adapter body may force the flexible silicone tubular body located on at least a portion of the exterior surface thereof against the interior wall of the cored hole, effectuating an air-tight, or almost air-tight, seal between the cored slab and the device. Exemplary embodiments of the adapter body may then be connected to a portion of the sampling tubing via an air-tight barbed fitting.

As mentioned above, it is also possible to manually install the adapter body devices and accouterments within the foundation of a home, building or other surface that contains a foundation made of concrete or similar substance. Whether designed for manual or automatic operation, devices, as well as those of the present disclosure, may be generally associated with an automatic soil fluid reading device (not shown). Such a soil fluid reading device is operative to automatically read the VOC levels of the native material 400 such as soil and/or gravel backfill 300 contained under the foundation wherein such devices are installed, such as depicted in FIGS. 4-5b.

FIG. 7 illustrates another exemplary embodiment of an adapter body 500. In this embodiment, the adapter body 500 has a first barbed end 505 and second barbed end 510. The adapter body 500 also has a male threaded collar 515 separating the first barbed portion 505 and the second barbed portion 510. A raised end 520 is provided at the distal end of the second barbed portion 510. As discussed herein, the first barbed portion 505 is sized and adapted to facilitate a connection between the adapter body 500 and a fluid sampling and/or measuring device (not shown). The second barbed portion 510 is sized and adapted for insertion into a tube 80. The adapter body 500 may have a unitary design or it may be constructed of modular sections. A modular construction would allow the first 505 and second 510 barbed portions and the threaded collar 515 to be changed to accommodate different sized components, thereby giving the adapter body 500 greater flexibility. The adapter body 500 may be made of brass or other material sufficiently strong to withstand the installation and extraction process. To allow soil fluid samples to be taken, the adapter body 500 has an internal passageway through which the soil fluid may travel.

The adapter body 500 is to be installed and extracted using an exemplary embodiment of a tool 600. FIG. 8 illustrates another exemplary tool 600 used for the installation and extraction of the adapter body 500. As shown, a tool 600 has a T-shaped body. The tool 600 includes a stem portion 610 and a handle portion 615. As shown in FIG. 8, the stem 610 has a first end 620 and second end 625. The second end 625 intersects the handle 615 so that the stem portion 610 extends substantially perpendicular from the handle 615. The first end 620 of the stem portion 610 is threaded and has an extraction cavity 630 therein. The threaded portion 640 of the first end 620 is a predetermined length sufficient for extraction of the adapter body 500, as will be discussed herein. To install the adapter body 500, the handle has at least one installation cavity 635 therein. As shown in FIG. 8, the installation cavity 635 is adapted to accommodate the first barbed end 505 of the adapter body 500.

To install the adapter body 500 using the tool 600, the first barbed end 505 is inserted into the installation cavity 635 in the handle 615. The tool 600 rests on a surface created by the threaded collar 515. A mallet or other device is then used to strike the end of the handle 615 opposite of the installation cavity 635 in order to force the adapter body 500 into the drilled core (as shown in FIG. 8). After installation of the adapter body 500, the tool 600 is simply removed from the adapter body 500 and the adapter body 500 is connected to a fluid sampling and/or measuring device.

A typical extraction of the adapter body 500 is illustrated in FIGS. 9 and 10. The threaded portion 640 of the first end 620 of the stem 610 is threaded into the coupling 700. The coupling 700 is threaded completely onto the pre-determined length of the threaded portion 640. The tool 600 is then used to thread the coupling 700 onto the threaded collar 515 of the adapter body 500. The coupling 700 can be threaded onto the adapter body 500 then the tool 600 may be threaded into the coupling 700.

To extract the adapter body 500 from the core, a user continues to turn the tool 600. Due to the threaded connection between the adapter body 500 and the coupling 700, the adapter body 500 is forced upward into the coupling 700. As the adapter body 500 is raised upward as a result of the rotational motion of the tool 600, the first barbed portion 505 of the adapter body 500 is inserted into the extraction cavity 630. This enables the adapter body 500 to be moved upward without the need to readjust the tool 600. Once the threaded collar 515 comes into contact with the first end 620 of the tool 600, the tool 600 can be used to lift the adapter body 500 from the drilled core.

In still other exemplary embodiments, rather than having a male threaded portion at the first end 620, the first end may have a female threaded portion (not shown in the figures). The female threaded portion may be sufficiently sized to be threaded onto the threaded collar 515 of the adapter body 500. In this embodiment, the need for a coupling 700 may be avoided.

After the adapter body 500 is installed, a covering 800 may be used to cover the hole created and to protect the adapter body 500. As illustrated in FIG. 11, the covering 800 includes a threaded portion 805, a cavity 810, a flange 815, and slotted portion 820. FIG. 12 further illustrates the exemplary covering 800 joined with the adapter body 500. As shown, the covering 800 is lowered onto the adapter body 500 so that the first barbed portion 505 is recessed within the cavity 810. To secure the covering 800, the threaded portion 805 of the covering 800 is threaded over the threaded portion 515 of the adapter body 500. The proper covering 800 fit results in the flange 815 of the covering 800 resting atop and being drawn to the surface of the material in which the adapter body 500 rests. To fully tighten down the covering 800, a screwdriver or other similar device may be used in the slotted portion 820.

To stand up to wear and tear, the covering 800 may be constructed from metal or other materials that are strong enough to protect the adapter body 500. Before the covering 800 is applied to the adapter body 500, a cap (not shown in the figures) may be placed over the first barbed portion 505 to prevent debris from entering the adapter body 500. Although the slotted portion 820 shown is for a spanner screwdriver, it also designed to accommodate flat, Phillips, and hex head screwdrivers as well as other tools.

While the advent of the devices generally described above has largely brought with it vastly improved techniques to the field sub-slab soil fluid collection, sampling and analysis, recent advances in the field have developed a surprising increase in demand for soil fluid collection and analysis at points beneath the slab. While preferred techniques are viewed as superior in that they, for example, provide reduced or eliminated leakage, are unobtrusive with respect to the interior of a building when installed, and have dramatically reduced the cost and difficulties of installation over previously-used devices, they have been found impractical to use in connection with other, external sampling devices placed within the sampling hole or used to collect samples at points beneath the slab.

There is also a desire in the field for the ability to collect for analysis samples of sub-slab soil fluid at a source that lies beneath the slab itself, or coincident with or adjacent to the base of the slab. It has also been discovered that sub-slab soil fluid collection at points at or beneath the bottom surface of the slab is often impractical with prior art devices due to contamination, blockage and clogging, and moisture collection concerns.

A system 1000, depicted in FIG. 16 and described hereinafter, also provides certain improvements in the collection, sampling and analysis process of sub-slab soil fluid in view of repeated sampling that often occurs at multiple locations. For instance, foundation slab thicknesses may often vary from location to location to such an extent that those in the field must either obtain multiple sizes of the prior art devices or obtain often unobtainable knowledge of slab thickness prior to coring, in order to align the ingress opening of the device at a precise position relative to the top or bottom surface of the slab involved. To overcome these and other drawbacks with the current art, the present disclosure utilizes a receptacle 900 for sampling and/or measuring devices. The receptacle 900 allows for passive testing of a fluid.

As will be explained in further detail below, exemplary embodiments of the adapter body have a length and proximal and distal ends, and are generally provided with a first barbed portion disposed at the proximal end of the adapter body, a second barbed portion disposed at the distal end of the adapter body, a collar portion disposed between the first barbed portion and the second barbed portion, and an internal cavity having an interior surface and passing through the length of the adapter body. The aforementioned features are similar in function and variety to those described above in exemplary adapter bodies, for example adapter bodies 15 and 500 shown in FIGS. 1 and 7, respectively. Exemplary adapter bodies used in the system 1000, however, also include at least a coupling portion having an internal thread disposed on the interior surface and extending longitudinally thereon from the distal end of the adapter body.

FIG. 13 illustrates an embodiment of a receptacle 900 for a sampling and/or measuring device. The receptacle 900 may be configured to cooperate with the adapter bodies 15, 500 shown in FIGS. 1 and 7, respectively. It is understood, however, that the receptacle 900 may be employed with other components, devices, and systems for facilitating a sampling and/or measuring of fluid such as sub-slab soil gas, as desired. In some embodiments, the receptacle 900 comprises a housing portion 902 (depicted in FIGS. 14A-14H) and a sieve portion 952 (depicted in FIGS. 15A-15E).

Referring to FIGS. 14A-14H, the housing portion 902 is shown having a first end 904, a second end 906, and an intermediate segment 907 therebetween. An internal cavity 908 extends longitudinally through the housing portion 902 from an opening 909 formed in the first end 904 to an opening 910 formed at the second end 906. In preferred embodiments, a diameter D1 of the cavity 908 formed in the first end 904 is generally commensurate with or equal to the diameter of the internal cavity of the adapter body. A diameter D2 of the cavity 908 formed in the second end 906 and the intermediate segment 907 is generally equal to or greater than a diameter of a fluid sampling and/or measuring device 2 (e.g., see FIG. 16).

The housing portion 902 is shown having a first coupling feature 912 that is complimentary with the adapter bodies 15, 500. The housing portion 902 preferably utilizes an external thread provided at the first end 904 as the first coupling feature 912, which is adapted for complimentary threaded retention within a coupling portion of the adapter body.

A second coupling feature 914 may also be provided on the housing portion 902. The housing portion 902 preferably utilizes an external thread provided at the second end 906 as the second coupling feature 914, which is adapted for complimentary threaded retention within the sieve portion 952 of the receptacle 900.

It is understood that various other coupling features may be employed to connect the housing portion 902 of the receptacle 900 to the adapter body.

The housing portion 902 may further include an external engaging region (not depicted). The external engaging region generally provides a geometry suitable for engagement with a hand tool such as a wrench or other tool that is useful for assembling and disassembling the receptacle 900 from the adapter body or other components of the system. In one embodiment, the external engaging region is disposed between the first end 904 and the second end 906. The external engaging region may be of substantially circular shape with a pair of opposed sides that are substantially parallel to one another. The opposing sides may be described as secants of the substantially circular shape. In other embodiments, the external engaging region may be substantially hexagonal or square in cross-sectional shape, or other such geometries suitable for use with a wrench or other tools to provide a mechanical advantage.

Turning now to FIGS. 15A-15E, an embodiment of the sieve portion 952 is illustrated. The sieve portion 952 permits a flow of a fluid into the receptacle 900, while maintaining a position of the sampling and/or measuring device 2 in the internal cavity 908.

Referring to these figures, the sieve portion 952 is shown having a first end 954 and a second end 956 generally defining a length therebetween. An internal cavity 958 extends longitudinally through the extension 952 from an opening 959 formed in the first end 954 to an opening 960 formed in the second end 956. In preferred embodiments, a diameter D1 of the cavity 958 formed in the first end 954 is generally commensurate with or equal to a diameter of the second end 906 of the housing portion 902 and a diameter D2 of the cavity 958 formed in the second end 956 is generally commensurate with or equal to the diameter D1 of the internal cavity 908 of the housing portion 902 (e.g., see FIG. 16).

In some embodiments, the sieve portion 952 may be releaseably coupled to the housing portion 902. It is understood, however, that in other embodiments the sieve portion 952 may be fixedly coupled to the housing portion 902. As illustrated, the first end 954 of the sieve portion 952 includes at least one coupling feature 961. In a preferred embodiment, the first end 954 of the sieve portion 952 is provided with internal threads formed on an interior surface of the internal cavity 958 as the coupling feature 961. The internal threads extend longitudinally on the interior surface, and are configured to threadably retain the second coupling feature 914 (e.g., the external threads) provided on the second end 906 of the housing portion 902.

It is understood that various other coupling features may be employed to connect the sieve portion 952 to the housing portion 902 of the receptacle 900.

In some embodiments, the sieve portion 952 may further include one or more lateral openings 962, in addition to the opening 960, each intersecting with the internal cavity 958 to provide alternate pathways through which sub-slab soil fluid may enter the receptacle 900, and thereby the system. It is understood that the sieve portion 952 may be formed without the opening 960 in various embodiments of the present disclosure. Accordingly, the first end 954 of the sieve portion 952 may be an open end and the second end 956 thereof may be a closed end. In preferred embodiments, six lateral openings 962 are formed entirely through the sieve portion 952 and intersecting with the internal cavity 958 to provide a total of twelve lateral inlets, although more or less may be provided without departing from the system.

In some embodiments, the sieve portion 952 of the receptacle 900 may further include an external engaging region (not depicted). The external engaging region generally provides a geometry suitable for engagement with a hand tool such as a wrench or other tool that is useful for assembling and disassembling the sieve portion 952 from the housing portion 902 of the receptacle 900 or other components of the system. In one embodiment, the external engaging region is disposed between the first end 954 and the second end 956. The external engaging region may be of substantially circular shape with a pair of opposed sides that are substantially parallel to one another. The opposing sides may be described as secants of the substantially circular shape. In other embodiments, the external engaging region may be substantially hexagonal or square in cross-sectional shape, or other such geometries suitable for use with a wrench or other tools to provide a mechanical advantage.

With regards to FIG. 16, a sectional view of an exemplary embodiment of a fluid sampling and/or measuring system 1000 in use in connection with an exemplary structure or slab 200 and material or substrate 300 (e.g., backfill) in illustrated. The system 1000 includes an improved adapter body 1002 having a length and proximal 1004 and distal 1006 ends. The adapter body 1002 generally includes a first barbed portion 1008 formed at the proximal end 1004 and a second barbed portion 1010 formed at the distal end 1006. A collar portion 1012 is formed between the first barbed portion 1008 and the second barbed portion 1010, and an internal cavity 1014 having an interior surface 1016 passes through the length of the adapter body 1002.

The adapter body 1002, further includes a coupling feature 1018 provided at the distal end 1006 of the adapter body 1002. In preferred embodiments, the coupling feature 1018 of the adapter body 1002 are internal threads formed on the interior surface 1016 of the internal cavity 1014, extending longitudinally thereon from the distal end 1006 of the adapter body 1002 to about the second barbed portion 1010. The coupling feature 1018 may be adapted for complimentary threaded retention of an coupling feature 912 of the receptacle 900 for a sampling and/or measuring device, for instance the housing portion 902 as shown in FIG. 16 and described in more detail in connection with FIGS. 14A-14H.

The receptacle 900 and system 1000 may thus be used to sample sub-slab soil fluid with increased efficiency and extensibility, and further reduces the intrusion of such sampling activities into the day-to-day operations being conducted in any given sampling site.

The exemplary embodiments were chosen and described in order to explain some of the principles of the present disclosure so that others skilled in the art may practice the present disclosure. While certain embodiments of the present disclosure are described in detail above, the scope of the present disclosure is not to be considered limited by such disclosure, and modifications are possible without departing from the spirit of the present disclosure.

1. A receptacle, comprising:
a housing portion having a cavity configured to receive at least one sampling and/or measuring device for a sub-structure fluid, wherein an entirety of the housing portion is located at or below an exposed surface of a structure when the receptacle is disposed in the structure.

2. The receptacle of claim 1, wherein the cavity is configured to permit a flow of the sub-structure fluid therethrough.

3. The receptacle of claim 1, wherein the cavity has a first diameter and a second diameter, and wherein the second diameter is greater than the first diameter.

4. The receptacle of claim 3, wherein the first diameter is substantially equal to a diameter of a cavity formed in an adapter body.

5. The receptacle of claim 1, wherein the housing portion includes a first end, a second end, and an intermediate segment formed therebetween.

6. The receptacle of claim 5, wherein the cavity extends along a longitudinal axis of the housing portion from the first end to the second end.

7. The receptacle of claim 5, wherein at least one of the first end and the second end is provided with one or more coupling features.

8. The receptacle of claim 5, wherein the first end is configured to be coupled to an adapter body and the second end is configured to be coupled to a sieve portion of the receptacle.

9. A system for facilitating a sampling and/or measuring of a sub-structure fluid, comprising:
a receptacle including a housing portion and a sieve portion configured to be coupled to the housing portion, the housing portion having a cavity configured to receive at least one sampling and/or measuring device for the sub-structure fluid, wherein an entirety of the housing portion is located at or below an exposed surface of a structure when the receptacle is disposed in the structure.

10. The system of claim 9, wherein the cavity of the housing portion has a first diameter and a second diameter, and wherein the second diameter is greater than the first diameter.

11. The system of claim 10, wherein the first diameter is substantially equal to a diameter of a cavity formed in an adapter body.

12. The system of claim 9, wherein the housing portion includes a first end, a second end, and an intermediate segment formed therebetween.

13. The system of claim 12, wherein the cavity extends along a longitudinal axis of the receptacle from the first end to the second end of the housing portion.

14. The system of claim 12, wherein at least one of the first end and the second end is provided with one or more coupling features.

15. The system of claim 12, wherein the first end is configured to be coupled to an adapter body and the second end is configured to be coupled to the sieve portion.

16. The system of claim 9, wherein the sieve portion includes a cavity formed therein to permit a flow of the sub-structure fluid therethrough.

17. The system of claim 9, wherein the sieve portion includes one or more lateral openings formed therein to permit a flow of the sub-structure fluid therethrough.

18.-20. (canceled)

21. A system for facilitating a sampling and/or measuring of a sub-structure fluid, comprising:
an adapter body; and
a receptacle coupled to the adapter body, the receptacle including a housing portion having a cavity configured to receive at least one sampling and/or measuring device for the sub-structure fluid, wherein an entirety of the housing portion is located at or below an exposed surface of a structure when the receptacle is disposed in the structure.

22. The system of claim 21, wherein the receptacle further comprises a sieve portion coupled to the housing portion.

23. The system of claim 21, wherein an end of the adapter body is at or below the exposed surface of the structure when the receptacle is disposed in the structure.
